(19) United States
(12) Patent Application Publication
(10) Pub. No.: US 2025/0161878 A1
(43) Pub. Date: May 22, 2025

(54) PORTABLE AIR PURIFIER FOR PROVIDING STERILIZING AND DEODORIZING FUNCTIONS

(71) Applicants: Hanon Systems, Daejeon (KR); HYUNBO CORPORATION, Chungcheongnam-do (KR)

(72) Inventors: Su Jin Woo, Daejeon (KR); Ji-Yong Park, Daejeon (KR); Sung Ro Go, Chungcheongbuk-Do (KR)

(21) Appl. No.: 18/840,109

(22) PCT Filed: Jul. 7, 2023
(86) PCT No.: PCT/KR2023/009661

§ 371 Date: Aug. 21, 2024

(30) Foreign Application Priority Data
Jul. 14, 2022 (KR) 10-2022-0087000

(51) Int. Cl. B01D 53/88; A61L 9/20; B01D 53/00; B01D 53/86

(52) U.S. Cl. CPC B01D 53/885; A61L 9/205; B01D 53/007; B01D 53/8696; A61L 2209/12; B01D 2255/20707; B01D 2255/20776; B01D 2257/90; B01D 2257/91; B01D 2258/06; B01D 2259/4541; B01D 2259/4566; B01D 2259/802

(57) ABSTRACT

The present invention relates to an air purifier, and more particularly, to a portable or vehicle-mounted air purifier. The present invention is advantageous for effectively capturing and sterilizing airborne virus particles by generating OH radicals with sterilizing capabilities through the application of a light source and a photocatalyst containing a light-responsive material.

### TECHNICAL FIELD

The present invention relates to an air purifier, and more particularly, to a portable or vehicle-mounted air purifier.

### BACKGROUND ART

Indoor air contains various biological contaminants, such as influenza viruses, coronaviruses, methicillin-resistant Staphylococcus aureus (MRSA), mold, allergens (e.g., dust mite carcasses or excrement, pet-derived allergens), and air pollutants like active oxygen, all of which are harmful to human health. Particularly in enclosed indoor spaces such as car interiors and hospital rooms, the concentration of these pollutants is higher, leading to increased cases of harm caused by indoor air contaminants. In the wake of the COVID-19 pandemic, there has been a global surge in interest in indoor space sterilization.

To address these concerns, numerous air purifiers capable of sterilization and deodorization have been developed. Many of these utilize a dry filter method based on high-efficiency particulate air (HEPA) filters. While HEPA filters are highly efficient at removing fine dust, their sterilization effect on airborne microorganisms is very low, necessitating additional sterilization components that increase manufacturing costs, and with prolonged use, particles like fine dust accumulate on the filter, reducing air purification efficiency and potentially allowing microorganisms to breed. Additionally, frequent filter replacements incur ongoing expenses, and the dense structure of filters limits their capacity to process large volumes of air effectively.

### DISCLOSURE

### Technical Problem

The present invention has been conceived to solve the above problems and it is an object of the present invention to provide a portable air purifier capable of improving sterilization and deodorization functions by effectively capturing and sterilizing virus particles floating in the air through the application of a light source and a photocatalyst containing a light-responsive material, thereby generating OH radicals.

### Technical Solution

In order to accomplish the above objects, a portable air purifier with sterilization and deodorization functions according to an embodiment of the present invention includes a substrate unit including at least one light source, at least one photocatalyst configured to receive light from the light source, a photocatalyst unit configured to secure the photocatalyst and including an inner housing having one side to which, at the center, one end of the substrate unit is attached, a central housing configured to secure and support the photocatalyst, and a fan coupled to one end of the central housing and configured to discharge the air passing through the photocatalyst to the outside.

In addition, the at least one light source is arranged at predetermined intervals in the direction in which the substrate unit extends.

In addition, the inner housing includes a holder on one side into which one end of the photocatalyst is inserted, the substrate unit and the photocatalyst are coupled to one side of the inner housing, and the photocatalyst extends a predetermined length from one side of the inner housing.

In addition, the inner housing further includes, on the other side, an insertion portion in the form of a protrusion and a recess or groove configured to fit into a vehicle cup holder.

In addition, the central housing, at the other end, includes a photocatalyst support coupled to the other end or side of the photocatalyst, and the photocatalyst support, on one side, has a slit corresponding in shape to a side edge of the photocatalyst in order for the photocatalyst to be inserted.

In addition, the portable air purifier further includes a lower housing configured to enclose and protect the sides of the photocatalyst unit and the central housing, wherein the lower housing includes an inlet hole on the side through which external air enters.

In addition, the inlet hole is formed at a position corresponding to the photocatalyst.

In addition, the lower housing includes a non-slip pad on one end surface with a coefficient of friction greater than a predetermined value.

In addition, the lower housing, at the other end, is coupled to have a predetermined distance from the other end surface of the inner housing.

In addition, the portable air purifier further includes a control unit configured to control the intensity of the fan, wherein the fan includes a lamp attached to one side, the control unit classifies the intensity of the fan into sections, stores unique color information for each section, and controls the lamp to emit the unique color corresponding to a control value of the intensity of the fan.

In addition, the portable air purifier further includes an upper housing configured to enclose and protect the sides of the photocatalyst unit and central housing and coupled to the lower housing on one side, wherein the upper housing includes a baffle formed at a predetermined distance from the fan to direct the discharge of the internal air propelled by the fan.

In addition, the upper housing has a larger inner diameter at the other end than the average inner diameter of the portion where the baffle is formed.

### Advantageous Effects

The portable air purifier of the present invention, configured as described above, is advantageous in terms of effectively capturing and sterilizing airborne virus particles by generating OH radicals with sterilizing capabilities through the application of a light source and a photocatalyst containing a light-responsive material.

### DESCRIPTION OF DRAWINGS

FIG. 1 is an exploded view illustrating a portable air purifier with sterilization and deodorization functions according to the present invention;

FIG. 2 is a side view illustrating the operation of a portable air purifier with sterilization and deodorization functions according to the present invention;

FIG. 3 is a perspective view illustrating a substrate unit according to the present invention;

FIG. 4 is a perspective view illustrating a photocatalyst unit according to the present invention;

FIG. 5 is an exploded perspective view illustrating the coupling relationship between a photocatalyst unit and a central housing according to the present invention;

FIG. 6 is a side view illustrating a lower housing according to the present invention;

FIG. 7 is a partial perspective view illustrating a fan according to the present invention;

FIG. 8 is a partial perspective view illustrating an upper housing according to the present invention; and

FIG. 9 is a side view illustrating the coupling relationship between an upper housing and a fan.

### MODE FOR INVENTION

In the following, the technical aspects of the present invention will be described in more detail with reference to the accompanying drawings. Prior to this, the terms and words used in the following specification and claims should not be construed in a limited sense to their usual or dictionary meanings but should be interpreted according to the meanings and concepts that conform to the technical ideas of the present invention, based on the principle that the inventor can appropriately define the terms to best describe their invention.

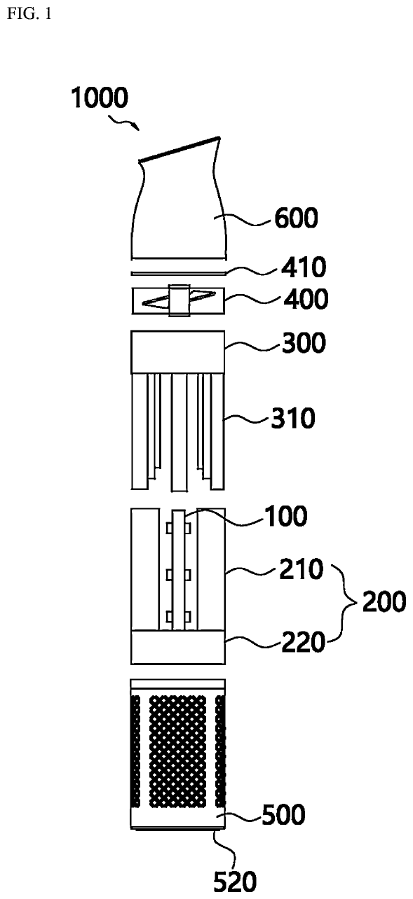

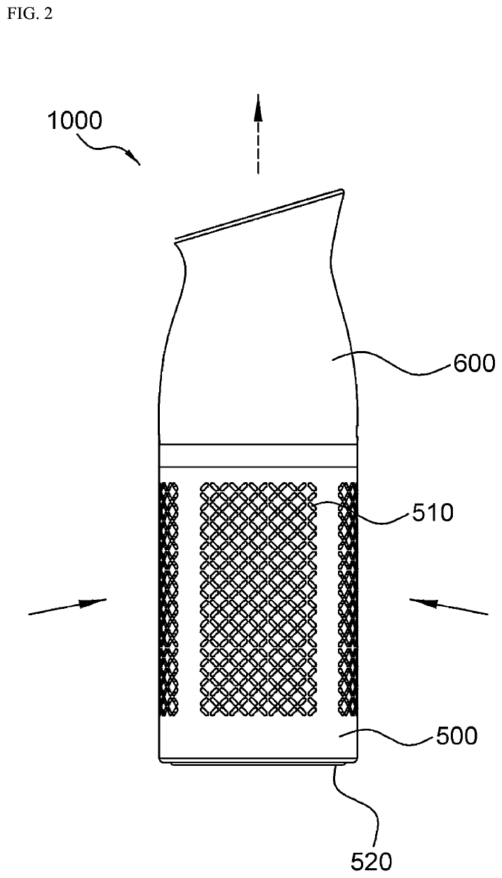

Hereinafter, a description is made of the portable air purifier 1000 with sterilization and deodorization functions according to the present invention in detail with reference to FIGS. 1 and 2.

As shown in FIG. 1, the portable air purifier 1000 with sterilization and deodorization functions according to the present invention may include a column-shaped substrate unit 100 that includes at least one light source 110, and a photocatalyst unit 200 that is irradiated with light from the substrate part 100. The photocatalyst unit 200 may include at least one photocatalyst 210 irradiated with light from the light source 110, and an inner housing 220 that secure the photocatalyst 210 while one end of the substrate unit 100 is attached to the center of one side. The photocatalyst 210 may include a light-responsive materials such as TiO₂ and WO₃.

The portable air purifier 1000 with sterilization and deodorization functions according to the present invention may include a central housing 300 that secures and supports the photocatalyst unit 200, and a fan 400 attached to one side of the central housing 300 to discharge air passing through the photocatalyst 210 to the outside. Mounting the fan 400 on the central housing 300 positions the fan at the center of the portable air purifier 1000 with sterilization and deodorization functions, ensuring stable airflow for both the intake of external air and the discharge of internal air.

Furthermore, the purifier may include an upper housing 600 and a lower housing 500 that enclose and protect the outer surface of the central housing 300 and the photocatalyst unit 200. In this configuration, one end of the upper housing 600 and the other end of the lower housing 500 may be joined together, the one end of the upper housing 600 may be the lower end of the upper housing based on FIG. 1, and the other end of the lower housing 500 may be the upper end of the lower housing based on FIG. 1. In this way, the upper housing 600 and lower housing 500 are joined to form an internal space that accommodate the central housing 300, fan 400, photocatalyst unit 200, and substrate unit 100.

As depicted in FIG. 2, the portable air purifier 1000 with sterilization and deodorization functions, configured as described above in the present invention, generates airflow from the lower housing 500 side to the upper housing 600 side by rotating the fan 400, allowing external air to enter through the inlet holes 510 formed in the lower housing 500, pass through the photocatalyst unit 200 for sterilization and deodorization, and then be discharged back outside through the fan 400 and the upper housing 600. Accordingly, the portable air purifier 1000 with sterilization and deodorization functions according to the present invention is capable of sterilizing microorganisms such as mold spores and viruses floating in the air of the indoor space where it is deployed.

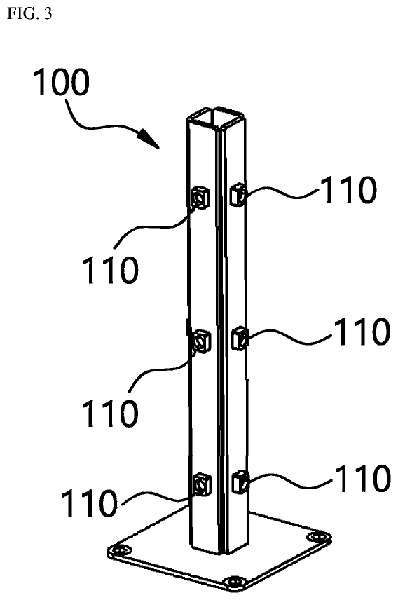

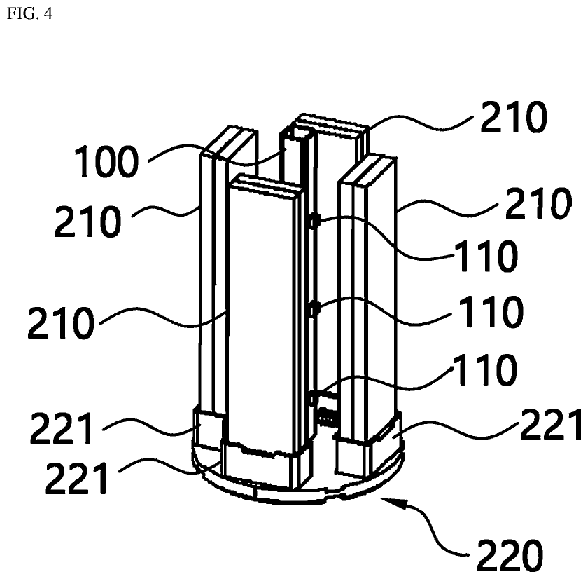

Hereinafter, descriptions are made of the substrate unit 100, the photocatalyst unit 200, and the coupling relationship thereof according to the present invention in more detail with reference to FIGS. 3 and 4.

As shown in FIG. 3, the substrate unit 100 may include the light sources 110 arranged on the side thereof. As described above, the substrate unit 100 may have a column shape element elongated in one direction, and the light sources 110 may be arranged at predetermined intervals along the direction of elongation of the substrate unit 100.

Additionally, the substrate unit 100 may include a support plate attached to one end. The support plate may be a flat surface perpendicular to the direction of elongation of the substrate unit 100. Attaching the support plate to the center of the inner housing 220 allows the elongation direction of the substrate unit 100 to be perpendicular to the ground when the bottom surface of the lower housing 500 contacts the ground.

As shown in FIG. 4, the inner housing 220 may include holders 221 into which one end of the photocatalyst 210 is inserted. The holder 221 may include a socket configured to accommodate the photocatalyst 210, encompassing and securing not only one end surface of the photocatalyst 210 but also the side surfaces that contact the end face. Accordingly, the substrate unit 100 and the photocatalyst 210 may be coupled in a direction perpendicular to one side of the inner housing 220, which is parallel to the ground when the lower housing 500 rests on the ground. Thus, the photocatalyst 210 may extend a predetermined length in a direction perpendicular to the one side surface of the inner housing 220.

In this case, when the substrate unit 100 is formed as an n-sided column, each side of the substrate unit may be arranged in a one-to-one correspondence with a facing surface of a respective photocatalyst 210. In addition, when the substrate unit 100 extends in a cylindrical shape, the photocatalyst 210 may be formed in a cylindrical shape with an interior space the substrate unit 100 positioned at the center thereof. Alternatively, the photocatalyst 210 may be densely arranged so that a side of the light source 110 arranged on the side of the substrate unit 100 corresponds to one side of the photocatalyst 210. By arranging the photocatalyst 210 and the substrate unit 100 in this manner, the reaction efficiency of the photocatalyst 210 can be increased.

According to an embodiment, the inner housing 220, on the other side, may further include an insertion member in the form of a protrusion and a recess or groove that fits into a vehicle cup holder. The insertion member may be formed on the other side surface of the inner housing 220, and the shape of the insertion member may be flexibly adapted depending on the type of vehicle to which the portable air purifier 1000 with sterilization and deodorization functions according to the present invention is applied. The presence of the insertion member facilitates the easy application of the portable air purifier 1000 with sterilization and deodorization functions according to the present invention to vehicles, which represent a specialized indoor environment.

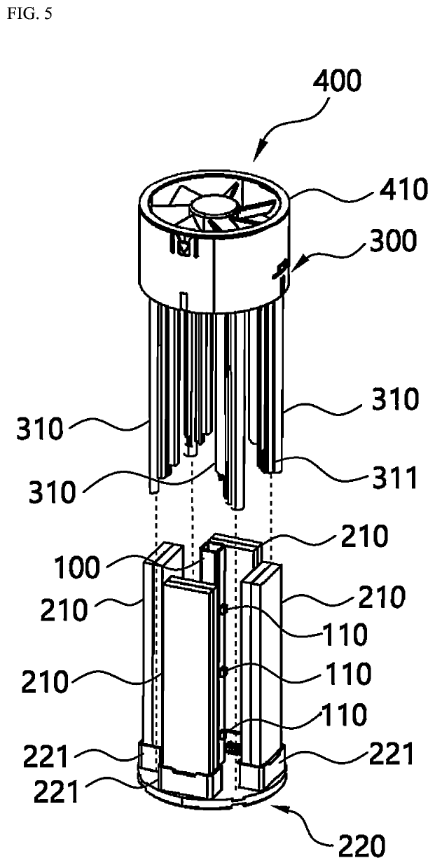

Hereinafter, a description is made of the coupling relationship of the substrate unit 200 and the central 300 according to the present invention in more detail with reference to FIG. 5.

As shown in FIG. 5, the central housing 300, at the other end, may include a photocatalyst support 310. The photocatalyst support 310 may be coupled with the other end or side of the photocatalyst 210. The photocatalyst support 310 may be formed by extending from the other end surface of the central housing 300 in a column shape and may be formed in the same number as the number of photocatalysts 210 and holders 221. In addition, the photocatalyst support 310, on one side, may include a slit 311 corresponding in shape to a side edge of the photocatalyst 210 in order for the photocatalyst 210 to be inserted.

That is, the photocatalyst 210 is inserted to the slit to mitigate the lateral vibration. As a result, improving the portability of the portable air purifier with stable support for the position of the photocatalyst 210 makes it possible to reduce inconvenience caused by light leakage from the light source 110 of the substrate unit 100 to the outside of the portable air purifier 1000 with sterilization and deodorization functions.

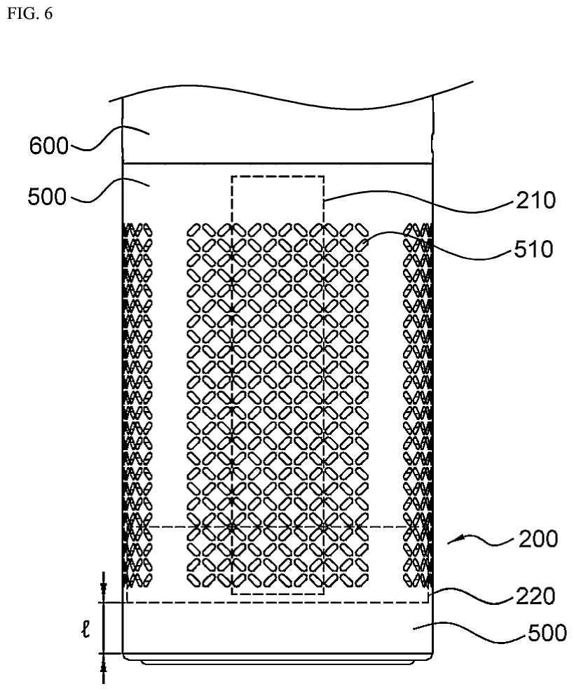

Hereinafter, a description is made of the coupling relationship between the lower housing 500 and other components according to the present invention in detail with reference to FIG. 6.

As shown in FIG. 6, the portable air purifier 1000 with sterilization and deodorization functions according to the present invention may further include the lower housing 500 that encloses and protects the sides of the photocatalyst unit 200 and central housing 300, the lower housing 500 including inlet holes 510 formed on the side for introducing external air. The inlet holes 510 may be formed at positions corresponding to the photocatalyst 210. This can increase the probability of external air passing through the photocatalyst 210. The inlet holes 510 may be formed in a consistent pattern with small fine holes arranged continuously.

In more detail, the inlet holes 510 may be formed in an elongated shape rather than a circular shape and arranged such that their elongated directions are perpendicular to each other, thereby forming a grid-like pattern across the entire set of inlet holes 510. Furthermore, such a grid-like pattern may be arranged only in specific areas on the side of the lower housing 500, which correspond to the photocatalyst 210 as described earlier, with each grid-shaped area formed at regular intervals and spaced apart from each other.

Furthermore, the lower housing 500 may include a non-slip pad 520 on one end surface with a coefficient of friction greater than a predetermined value. This helps prevent slipping when the one end surface of the lower housing 500 is positioned on the ground. Although the relatively heavy fan 400, substrate unit 100, and photocatalyst unit 200 are positioned in the center rather than at the bottom, the inclusion of a non-slip pad 520 may prevent the portable air purifier with sterilization and deodorization function according to the present invention from tipping over when the vehicle to which the air purifier is applied tilts during operation.

The lower housing 500, on one side, may further include an insertion member in the form of a protrusion and a recess or groove that fits into a vehicle cup holder. The shape of the insertion member may be flexibly adapted depending on the type of vehicle to which the portable air purifier 1000 with sterilization and deodorization functions according to the present invention is applied. The presence of the insertion member facilitates the easy application of the portable air purifier 1000 with sterilization and deodorization functions according to the present invention to vehicles, which represent a specialized indoor environment.

In addition, the lower housing 500 may be coupled to the inner housing 220 such that the other end of the lower housing has a predetermined distance from the other end surface of the inner housing. That is, the lower housing 500 may have a groove or protrusion inside to ensure that the inner housing 220 or central housing 300 is hooked and secured within the lower housing 500. The distance between the other end surface of the lower housing 500 and the other surface of the inner housing 220 may be determined based on the light irradiation angle of the light source 110 of the substrate unit 100. This may enhance the filtering efficiency.

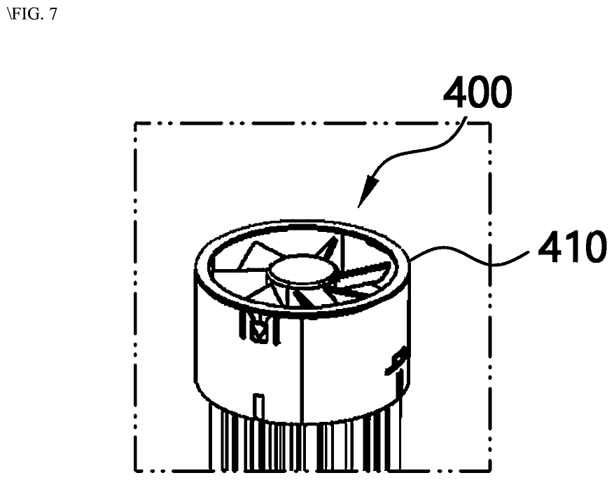

Hereinafter, descriptions are made of the fan 400 and the control unit for controlling the fan with reference to FIG. 7.

As shown in FIG. 7, the fan 400 may include a lamp 410 attached to one side thereof. The lamp 410 may be an LED lamp 410 and may irradiate light towards the side of the fan 400, i.e., in the direction of the upper housing 600. The portable air purifier 1000 with sterilization and deodorization functions according to the present invention may further include a control unit that adjusts the intensity of the fan 400, classifies the intensity of the fan into sections, stores unique color information for each section, and controls the lamp 410 to emit the unique color corresponding to the control value of the intensity of the fan 400. In order for the light of the lamp 410 to be visible to the user, the upper housing 600 may have apertures formed around the periphery through which light can pass. As a result, the user may intuitively observe the operation status and operating intensity (indicating indoor air cleanliness) of the portable air purifier 1000 with sterilization and deodorization functions according to the present invention. In addition, changing colors based on the operating intensity of the fan 400 can enhance aesthetics.

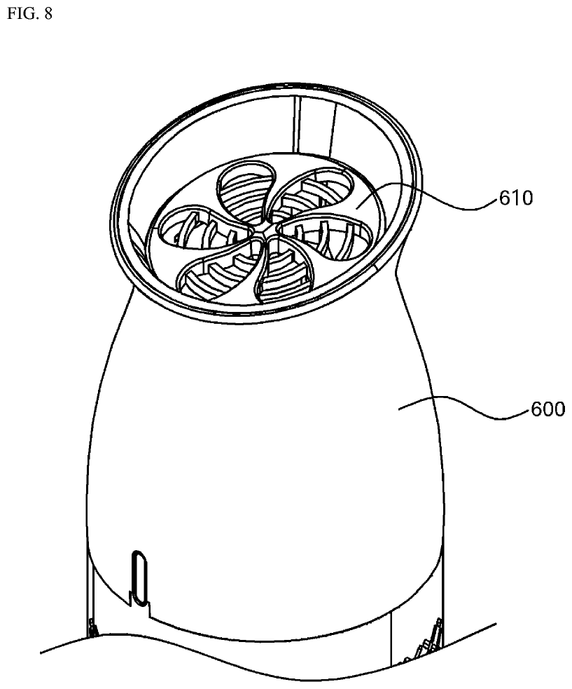

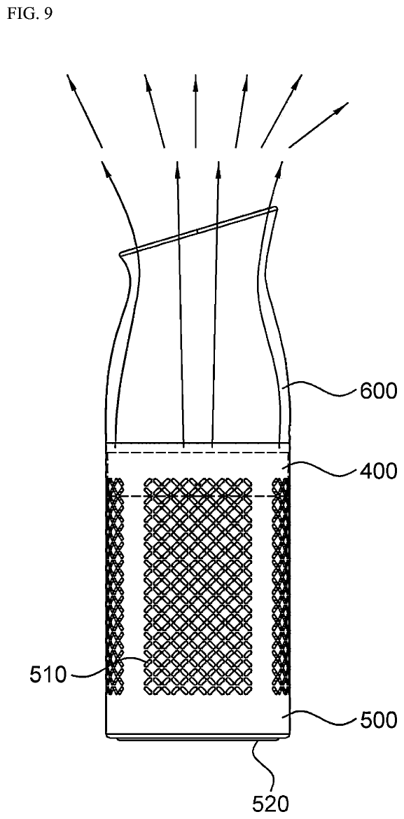

Hereinafter, a description is made of the upper housing 600 according to the present invention with reference to FIGS. 8 and 9.

As shown in FIG. 8, the portable air purifier 1000 with sterilization and deodorization functions according to the present invention may further include an upper housing 600 that is, at one end, coupled to the other end of the lower housing 500. As described above, the upper housing 600 may include apertures around its periphery to allow the light from the lamp 410 to pass through. In addition, the upper housing 600 may be open on the other side to allow the air propelled by the fan 400 to be discharged.

The upper housing 600 may also include a baffle 610 formed at a predetermined distance from the fan 400 inside. The baffle 610 may be assembled and designed to be detachable from the upper housing 600. The presence of the baffle 610 may protect the fan 400 from hazardous elements to the fan 400. In addition, the baffle 610 may enhance the aesthetic appeal by blending with the light from the lamp 410.

Furthermore, the upper housing 600 may be designed with a narrower inner diameter at the center, as shown in FIG. 9. In more detail, the inner diameter at the other side may be larger than the inner diameter of the portion where the baffle 610 is formed. As a result, the portable air purifier 1000 with sterilization and deodorization functions according to the present invention is capable of enhancing its aesthetic appeal when placed indoors by having a shape reminiscent of a vase. Furthermore, the internal air propelled by the fan 400 flows from a narrow area within the upper housing 600 to a wider area (the other end), allowing sterilized and deodorized air to spread into a larger area. This can facilitate sterilization and deodorization of indoor air.

The technical concept of the present invention should not be interpreted solely based on the above-described embodiments. It should be understood that various modifications and changes are possible within the scope of the claims without departing from the essence of the invention claimed in the claims. Therefore, such improvements and changes fall within the scope of protection of the present invention, as long as they are obvious to those skilled in the art.

1. A portable air purifier with sterilization and deodorization functions, the portable air purifier comprising:
a substrate unit comprising at least one light source;
at least one photocatalyst configured to receive light from the light source;
a photocatalyst unit configured to secure the photocatalyst and comprising an inner housing having one side to which, at the center, one end of the substrate unit is attached;
a central housing configured to secure and support the photocatalyst; and
a fan coupled to one end of the central housing and configured to discharge the air passing through the photocatalyst to the outside.

2. The portable air purifier of claim 1, wherein the at least one light source is arranged at predetermined intervals in the direction in which the substrate unit extends.

3. The portable air purifier of claim 1, wherein the inner housing comprises a holder on one side into which one end of the photocatalyst is inserted, the substrate unit and the photocatalyst are coupled to one side of the inner housing, and the photocatalyst extends a predetermined length from one side of the inner housing.

4. The portable air purifier of claim 1, wherein the inner housing further comprises, on the other side, an insertion portion in the form of a protrusion and a recess or groove configured to fit into a vehicle cup holder.

5. The portable air purifier of claim 1, wherein the central housing, at the other end, comprises a photocatalyst support coupled to the other end or side of the photocatalyst, and the photocatalyst support, on one side, has a slit corresponding in shape to a side edge of the photocatalyst in order for the photocatalyst to be inserted.

6. The portable air purifier of claim 1, further comprising a lower housing configured to enclose and protect the sides of the photocatalyst unit and the central housing,
wherein the lower housing comprises an inlet hole on the side through which external air enters.

7. The portable air purifier of claim 6, wherein the inlet hole is formed at a position corresponding to the photocatalyst.

8. The portable air purifier of claim 6, wherein the lower housing comprises a non-slip pad on one end surface with a coefficient of friction greater than a predetermined value.

9. The portable air purifier of claim 6, wherein the lower housing, at the other end, is coupled to have a predetermined distance from the other end surface of the inner housing.

10. The portable air purifier of claim 1, further comprising a control unit configured to control the intensity of the fan,
wherein the fan comprises a lamp attached to one side, the control unit classifies the intensity of the fan into sections, stores unique color information for each section, and controls the lamp to emit the unique color corresponding to a control value of the intensity of the fan.

11. The portable air purifier of claim 6, further comprising an upper housing configured to enclose and protect the sides of the photocatalyst unit and central housing and coupled to the lower housing on one side,
wherein the upper housing comprises a baffle formed at a predetermined distance from the fan to direct the discharge of the internal air propelled by the fan.

12. The portable air purifier of claim 11, wherein the upper housing has a larger inner diameter at the other end than the average inner diameter of the portion where the baffle is formed.
